(19) United States
(12) Patent Application Publication
(10) Pub. No.: US 2021/0395208 A1
(43) Pub. Date: Dec. 23, 2021

(54) AMORPHOUS COMPOUNDS OF FORMULA (I) AND AMORPHOUS COMPOUNDS OF FORMULA (I) SALTS

(71) Applicant: H. Lundeck A/S, Valby (DK)

(72) Inventors: Jens Kateb, Valby (DK); Matthias Manne Knopp, Valby (DK)

(73) Assignee: H. Lundeck A/S, Valby (DK)

(21) Appl. No.: 17/289,518

(22) PCT Filed: Oct. 28, 2019
(86) PCT No.: PCT/EP2019/079350

§ 371 Date: Apr. 28, 2021

(30) Foreign Application Priority Data
Oct. 29, 2018 (DK) PA201800783

(51) Int. Cl. C07D 241/04; A61K 47/32; A61K 47/34

(52) U.S. Cl. CPC C07D 241/04; C07B 2200/05; A61K 47/34; A61K 47/32

(57) ABSTRACT

The present invention relates to compositions comprising amorphous compounds of Formula (I) or amorphous compounds of Formula (I) salts and to processes for obtaining these. In particular, the invention relates to compositions comprising amorphous compounds of Formula (I) hydrogen fumarate and to processes for obtaining this. Further, the invention also relates to compositions comprising amorphous base of compounds of Formula (I) and to processes for obtaining this. The invention also relates to pharmaceutical compositions comprising amorphous compounds of Formula (I) or amorphous compounds of Formula (I) salts for treatment of psychiatric disorders such as schizophrenia, including Treatment Resistant Schizophrenia (TRS).

### FIELD OF THE INVENTION

The present invention relates to compositions comprising amorphous compounds of Formula (I) or amorphous compounds of Formula (I) salts and to processes for obtaining these. In particular, the invention relates to compositions comprising amorphous compounds of Formula (I) hydrogen fumarate and to processes for obtaining this. Further, the invention also relates to compositions comprising amorphous base of compounds of Formula (I) and to processes for obtaining this. The invention also relates to pharmaceutical compositions comprising amorphous compounds of Formula (I) or amorphous compounds of Formula (I) salts for treatment of psychiatric disorders such as schizophrenia, including Treatment Resistant Schizophrenia (TRS).

### BACKGROUND OF THE INVENTION

Throughout this application, various publications are referenced in full. The disclosures of these publications are hereby incorporated by reference into this application to describe more fully the state of the art to which this invention pertains.

WO9322293 describes substituted trans isomers of 1-piperazino-1,2-dihydroindene compounds and how to prepare such compounds. WO2012176066 describes deuterated 1-piperazino-3-phenyl-indane compounds of Formula (I)

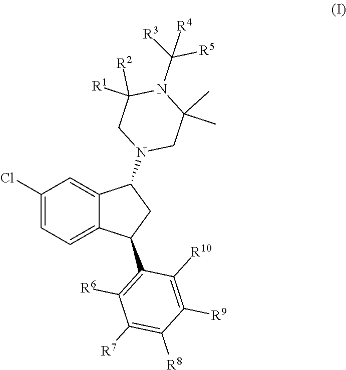

wherein, R1-R10 are independently selected from hydrogen or deuterium, wherein at least one of R1-R10 comprises at least about 50% deuterium, or a pharmaceutically acceptable acid addition salt thereof with activity at dopamine D1 and D2 receptors as well as the serotonin 5-HT2 receptors in the central nervous system. Compounds of Formula (I) can be prepared by processes described in WO2012176066 and this patent also describes additional steps for the preparation of the base and various salts of compounds of Formula (I).

WO2012176066 further disclose pharmaceutical compositions and medicaments comprising compounds of Formula (I) as active ingredients, and to the use of such compounds in the treatment of diseases in the central nervous system. Specifically, WO2012176066 disclose compound (Ia)

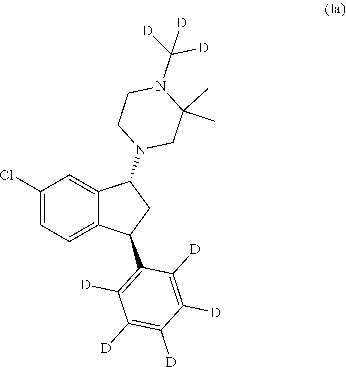 [SMILES: [2H]c1c([2H])c([2H])c([C@@H]2C[C@@H](N3CCN(C([2H])([2H])[2H])C(C)(C)C3)c3cc(Cl)ccc32)c([2H])c1[2H]]

In compound (Ia) eight hydrogen atoms have been isotopically substituted by deuterium atoms (denoted by “D” in the structure). This isotopic substitution reduces metabolism of the deuterated compound compared to the hydrogen substituted version. However, the majority of the physiochemical properties: e.g. crystallinity, polymorph selection, salt form selection, solubility in all vehicles etc. are expected to be similar for the hydrogen and the deuterated versions of any compounds (Gant, J. Med. Chem. 2014, 57, 3595-3611). Consequently, all compounds of Formula (I), regardless of the number of deuterated sites, are expected to have similar physiochemical properties. This similarity is also expected in regards to the preparation and stability of the amorphous forms of compounds of Formula (I).

As mentioned above the isotopic substitution facilitates a reduced metabolism and hence, deuterated compounds are anticipated to have higher bioavailability and less metabolite formation compared to the fully hydrogen substituted compounds. These characteristics are expected to allow lower doses to be administered to humans i.e. less burden to the entire body, e.g. the liver, and possibly a less frequent dosing.

WO2012176066 further discloses the use of a compounds of Formula (I) or a pharmaceutical composition comprising a compounds of Formula (I) in the treatment of psychosis, other diseases involving psychotic symptoms, psychotic disorders or diseases that present with psychotic symptoms e.g. schizophrenia.

Because the mechanism of atypical antipsychotics is not well understood, side effects associated with these drugs have been difficult to design around. Compounds of Formula (I) have the potential to accommodate this unmet need for additional antipsychotic therapies with reduced side effect and/or improved therapeutic profile relative to existing therapies.

In the future development of such potentially improved antipsychotic therapies, novel solid forms of compounds of Formula (I) with enhanced dissolution profiles would be beneficial; because the enhanced dissolution profile is expected to yield an even better bioavailability of the compounds of Formula (I). In this respect, amorphous materials generally offer interesting physio-chemical and pharmacological properties. Typically, amorphous materials will have a higher dissolution rate than the crystalline forms. These characteristics will generally lead to improved bioavailability of the pharmaceutical agent and enable novel formulation and dosing strategies e.g. rapid-onset formulations.

Unfortunately, obtaining an amorphous solid suitable for pharmaceutical formulation is often difficult, because the amorphous form is considered thermodynamically unstable and quickly converts partially or completely back to the more thermodynamically stable crystalline form. Hence, very few marketed drugs are available in amorphous form despite their obvious advantages.

It is the objective of the present invention to provide novel compositions comprising stable amorphous compounds of Formula (I) and amorphous compounds of Formula (I) salts. Such compositions have potential advantageous properties in terms of one or more of the following; bioavailability, pharmacokinetic profile, formulation properties or administration in relation to effective treatment of CNS disorders e.g. schizophrenia, including treatment resistant schizophrenia (TRS).

### SUMMARY OF THE INVENTION

The present invention relates to compositions comprising amorphous compounds of Formula (I) or amorphous compounds of Formula (I) salt. In general, it is acknowledged that amorphous materials with a low glass transition temperature is expected to recrystallize rapidly and hence, are not anticipated to be suitable candidates for further development in the pharmaceutical industry (Hancock et. al, Pharmaceutical research, 1995, 12(6): 799-806). The present invention relates to amorphous compounds, which in spite of their relative low glass transition temperature, do not recrystallize under ambient conditions and hence, constitute very stable amorphous solids.

The chemical structure of the compounds of Formula (I) are disclosed in WO2012176066, which describes deuterated 1-piperazino-3-phenyl-indane compounds and in WO9322293, which describes fully hydrogen substituted trans isomers of 1-piperazino-1,2-dihydroindene compounds.

In the present invention, the compounds of Formula (I) and compounds of Formula (I) salts are defined as compounds of Formula (I):

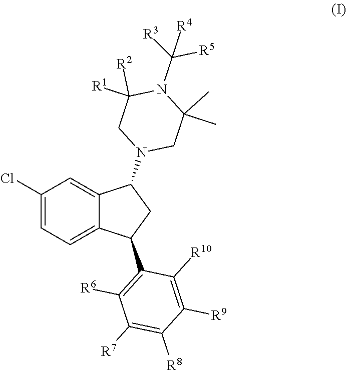

wherein, R1-R10 are independently selected from hydrogen or deuterium.

In a further aspect, the invention relates to compositions comprising amorphous compounds of Formula (I), wherein at least one of R1-R10 is deuterium, such as wherein R6-R10 are each deuterium and/or wherein R3-R5 are each hydrogen.

In a particular aspect, the present invention relates to compositions comprising the amorphous compound of Formula (Ia) or an amorphous compound of Formula (Ia) salt:

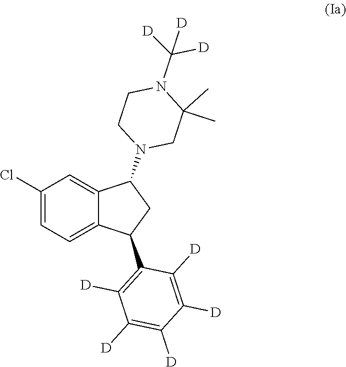 [SMILES: [2H]c1c([2H])c([2H])c([C@@H]2C[C@@H](N3CCN(C([2H])([2H])[2H])C(C)(C)C3)c3cc(Cl)ccc32)c([2H])c1[2H]]

In further aspects, the amorphous compound of Formula (Ia) may be the amorphous base and the amorphous compound of Formula (Ia) salt may be the amorphous compound of Formula (Ia) hydrogen fumarate.

In some aspects of the invention the compositions compromising amorphous compounds of Formula (I) or an amorphous compounds of Formula (I) salts, may further comprise one or more crystallization inhibitors, such as polymers, co-polymers, mesoporous silica etc. The composition may also be a pharmaceutical composition comprising one or more pharmaceutically acceptable excipients.

In a separate aspect, the invention relates to processes for obtaining amorphous compounds of Formula (I) or amorphous compounds of Formula (I) salts.

In yet another aspect, the present invention relates to amorphous compounds of Formula (I) or an amorphous compounds of Formula (I) salt or any composition comprising said amorphous materials for use in the treatment of psychotic disorders, in particular schizophrenia, including treatment resistant schizophrenia.

### Definitions

The term compound (I) is intended to cover both compounds of Formula (I) per see and compounds of Formula (I) salts unless otherwise specified.

The term “amorphous compound (I)” as used herein is intended to cover amorphous compounds of Formula (I) per see and amorphous compounds of Formula (I) salts. The term “amorphous” refers to a substantially non-crystalline solid form of compound (I). The non-crystalline form is characterized by the absence of a long-range order in the crystal lattice as determined, for instance, by X-ray powder diffraction (XRPD). The XRPD pattern of a non-crystalline and hence, essentially amorphous solid will be characterized by the absence of the Bragg peaks associated with the crystalline form, and the XRPD pattern takes the form of a diffuse halo.

The terms “substantially amorphous” as used herein refers to a solid amorphous compound (I) or amorphous compound (I) salts characterized by comprising at least 90 percent, preferably at least 95 percent, and even more preferably at least 97 percent amorphous solid form by total weight of the solid form.

The terms “actual glass transition temperature” and “calculated glass transition temperature” as used herein refers to the measured (actual) and theoretical (calculated) glass transition temperature associated with a specific compound of Formula (I) or compound of Formula (I) salt. The actual glass transition temperature is determined from the DSC thermogram for the amorphous material, which is exemplified in example 1 and depicted in FIG. 2. The calculated glass transition temperature is calculated as ⅔ of the melting temperature (in Kelvin) of the crystalline form. FIG. 1 illustrate DSC thermograms of crystalline forms of compound (Ia), from which the melting temperature can be determined. The calculation procedure for estimation of the glass transition temperature is described in example 1.

The term “crystallization inhibitor” as used herein refers to an agent or a substance, that reduces, delays or eliminates the formation of crystalline particles in the amorphous solid. Preferred examples of crystallization inhibitors are polymers, co-polymers, amino acids, mesoporous silicas and cyclodextrins.

The statement “reduces, delays or eliminates the formation of crystalline particles in the amorphous solid” as used herein refers to a composition comprising amorphous compound (I) or amorphous compound (I) salt and one or more crystallization inhibitors present in an amount that effectively preserves the substantially amorphous compound (I) or amorphous compound (I) salt in the amorphous state.

The term “amorphous solid dispersion” as used herein, refers to a solid dispersion comprising compound (I) or compound (I) salts and one or more polymers or co-polymers, wherein the solid dispersion is amorphous.

The terms “co-amorphous compositions” and “co-amorphous mixtures” as used herein is intended to describe amorphous compositions comprising compound (I) or compound (I) salts mixed with low molecular weight compound, e.g. amino acids.

The term “mesoporous silica” as used herein is intended to cover silica with a mesoporous morphology. Such materials encompass silicas with a particle size about 4-100 μm containing pores with a diameter of about 2-50 nm, the pore volume is about 0.50-1.75 cm³/g, and the surface area is usually 200 m²/g.

“Treatment resistant schizophrenia” as used herein, is meant to describe a pathological condition in a patient, wherein the patient lacks satisfactory clinical improvement despite two treatments with antipsychotics of adequate dose and duration.

The term “ambient conditions” as used herein refers to conditions with a temperature range of 15-35 degrees Celsius and a relative humidity range of 15-75 percent.

The term “salts” as used herein is intended to cover the group of salts comprising non-toxic, i.e. physiologically acceptable salts. These include salts formed with inorganic and/or organic acids such as hydrochloric acid, hydrobromic acid, phosphoric acid, nitrous acid, sulphuric acid, benzoic acid, citric acid, gluconic acid, lactic acid, maleic acid, succinic acid, tartaric acid, acetic acid, propionic acid, oxalic acid, maleic acid, fumaric acid, glutamic acid, pyroglutamic acid, salicylic acid, salicylic acid and sulfonic acids, such as methanesulfonic acid, ethanesulfonic acid, toluenesulfonic acid and benzene-sulfonic acid. Additional examples of useful acids to form suitable salts can be found e.g. in Stahl and Wermuth (Eds) “Handbook of Pharmaceutical salts. Properties, selection, and use”, Wiley-VCH, 2008.

In the present context, the term “therapeutically effective amount” of a compound means an amount sufficient to alleviate, arrest, partially arrest, remove or delay the clinical manifestations of a given disease and its complications in a therapeutic intervention comprising the administration of said compound. An amount adequate to accomplish this is defined as “therapeutically effective amount”. Effective amounts for each purpose will depend on the severity of the disease as well as the weight and general state of the subject. It will be understood that determining an appropriate dosage may be achieved using routine experimentation, which is all within the ordinary skills of a trained physician.

In the present context, “treatment” or “treating” is intended to indicate the management and care of a patient for the purpose of alleviating, arresting, partly arresting or delaying progress of the clinical manifestation of the disease. The patient to be treated is preferably a mammal, in particular a human being.

In the present context, the term “composition” is intended to describe mixture of two or more chemical substances. In the present invention one of these chemical substances must be an amorphous compound of Formula (I) or an amorphous compound of Formula (I) salt. Such composition may be a pharmaceutical composition if it further comprises one or more pharmaceutically acceptable excipient, which refers to pharmaceutical excipients including, but not limited to, fillers, anti-adherents, binders, coatings, colors, disintegrants, flavours, glidants, lubricants, preservatives, sorbents, sweeteners, solvents, vehicles and adjuvants.

The term “pharmaceutically acceptable excipient” refers to pharmaceutical excipients including, but not limited to, fillers, antiadherents, binders, coatings, colors, disintegrants, flavours, glidants, lubricants, preservatives, sorbents, sweeteners, solvents, vehicles and adjuvants.

The terms “animal,” “subject” and “patient” as used herein include, mammals (e.g., cats, dogs, horses, swine, etc.) and humans.

The term “isotopic substitution” as used herein means substituting one or more hydrogen in a parent compound by deuterium atoms. The places of substitution are denoted by “D” in Formula (Ia). It is recognized that elements are present in natural isotopic abundances in most synthetic compounds, and result in inherent incorporation of deuterium. However, the natural isotopic abundance of hydrogen isotopes such as deuterium is immaterial (about 0.015%) relative to the degree of stable isotopic substitution of compounds indicated herein. Thus, as used herein, designation of an atom as deuterium at a position indicates that the abundance of deuterium is significantly greater than the natural abundance of deuterium. Any atom not designated as a particular isotope is intended to represent any stable isotope of that atom, as will be apparent to the ordinarily skilled artisan.

The notation R1, R2, R3, R4, R5, R6, R7, R8, R9 and R10 may be used interchangeably with the notation R¹, R², R³, R⁴, R⁵, R⁶, R⁷, R⁸, R⁹ and R¹⁰.

### BRIEF DESCRIPTION OF FIGURES

FIG. 1 shows the DSC thermograms of the crystalline base of compound (Ia) (left) and the crystalline compound (Ia) hydrogen fumarate (right). The y-axis indicates heat flow measured in W/g (Exo Up) and the x-axis denotes temperature in degree Celsius. It is evident that the crystalline base of compound (Ia) has an endothermic peak around 60° C. corresponding to the melting of the crystals. The thermogram for the crystalline hydrogen fumarate of compound (Ia) shows an endothermic peak around 203° C. corresponding to the melting of the crystals.

FIG. 2 shows the DSC thermograms of the amorphous base of compound (Ia) (left) and the amorphous hydrogen fumarate of compound (Ia) (right). The y-axis indicates heat flow measured in W/g (Exo Up) and the x-axis denotes temperature in degree Celsius. The thermogram of the amorphous base of compound (Ia) shows a change in heat capacity around 5° C. corresponding to a glass transition. The thermogram of the amorphous hydrogen fumarate of compound (Ia) shows a change in heat capacity around 66° C. corresponding to a glass transition. For both compounds, there is no sign of recrystallization exotherms and melting endotherms above the glass transitions indicating high stability of the amorphous material.

FIG. 3 depicts the stability of amorphous compound (Ia) when stored at ambient conditions. The XRPD diffractograms of amorphous base of compound (Ia) prepared by melt quenching (left) is shown: immediately following preparation, after 1 week, 4 weeks, 9 weeks and 5 month, 8 months and 12 months (bottom-up). Similarly, the XRPD diffractograms of the amorphous hydrogen fumarate of compound (Ia) at the same time points are shown on the right. The X-axis is the diffraction angle (degree 2 theta) and the Y-axis is the intensity in counts.

FIG. 4 depicts the XRPD diffractograms of crystalline and amorphous compound (Ia) base (left) and crystalline and amorphous compound (Ia) hydrogen fumarate salt (right). The amorphous forms are prepared using melt quenching technique. Due to the lack of three-dimensional long-range order, the amorphous solids do not constructively diffract X-rays like the crystalline solids. Therefore, the XRPD diffractograms of the amorphous solids will have the shape of a broad diffuse halo, instead of well-defined peaks characterising the crystalline forms. The X-axis is the diffraction angle (degree 2 theta) and the Y-axis is the intensity in counts.

FIG. 5 shows the XRPD diffractograms of crystalline compound (Ia) as well as the resulting solids prepared by various techniques known to potentially produce amorphous solids. The compound (la) base solids are presented on the left and the compound (Ia) hydrogen fumarate solids are presented on the right. The XRPD diffractograms are depicted in the following order (from the top) crystalline, ball milling, melt quenching, spray drying and freeze drying. The X-axis is the diffraction angle (degree 2 theta) and the Y-axis is the intensity in counts.

FIG. 6 shows the DSC thermograms of the crystalline base and the crystalline hydrogen fumarate salt of compound (Ia) and selected polymers and co-polymers (left-crystalline base only; anionic methacrylate copolymer with an average molecular weight of 280.000 Da (Eudragit® FS100); polyvinyl caprolactam polyvinyl acetate-polyethylene glycol graft copolymer (Soluplus®) and Dimethylaminoethyl methacrylate, butyl methacrylate, and methyl methacrylate in a ratio of about 2:1:1 (Eudragit® EPO) (bottom-up), right—crystalline hydrogen fumarate only; Hypromellose Acetate Succinate (AQOAT AS MF); hydroxypropyl methylcellulose (Pharmacoat® 603); anionic methacrylate copolymer with an average molecular weight of 280.000 Da (Eudragit® FS100); polyvinyl caprolactam polyvinyl acetate-polyethylene glycol graft copolymer (Soluplus®); VP/VA copolymer: 60:40 linear random copolymer of N-vinyl-2-pyrrolidone and vinyl acetate (Plasdone™ S-630); Dimethylaminoethyl methacrylate, butyl methacrylate, and methyl methacrylate in a ratio of about 2:1:1 (Eudragit® EPO) and polyvinylpyrrolidone (Plasdone™ K12) (bottom-up)) recorded at a heating rate of 1° C./min.

### DETAILED DESCRIPTION OF THE INVENTION

The solid amorphous state of a material is characterized by the lack of three-dimensional long-range order found in crystalline solids. The amorphous form is further characterised by having a glass transition temperature (Tg). At temperatures below Tg the amorphous form exists in a solid state (termed glass) with a limited molecular mobility and at temperatures above Tg the amorphous form turns into a rubbery-like state and a sudden increment in molecular mobility that increases the probability of crystallization is obtained (Omar et. al. U K Journal of Pharmaceutical Biosciences, 2015, 3(6): 60-66).

Consequently, the glass transition temperature is an important parameter to consider when selecting suitable amorphous materials for pharmaceutical development. Generally, it is acknowledged that amorphous materials stored above Tg tend to recrystallize quickly, as sufficient molecular mobility exists within the system to facilitate nucleation and crystallization. For this reason, it is suggested to store amorphous materials at least 50 Kelvin below Tg to ensure stability (Hancock et. al, Pharmaceutical research, 1995, 12(6): 799-806). Conclusively, amorphous materials with low glass transition temperature are expected to recrystallize rapidly and hence, are not expected to constitute suitable candidates for further development in the pharmaceutical industry.

The glass transition temperature can be estimated as ⅔ of the melting temperature of the crystalline material (measured in Kelvin). This relationship is known as the “Beaman's rule” and applies to a wide range of both organic, inorganic, simple and polymeric materials. (Beaman, R. G. Journal of Polymer Science, 1952, 9(5): 470-472).

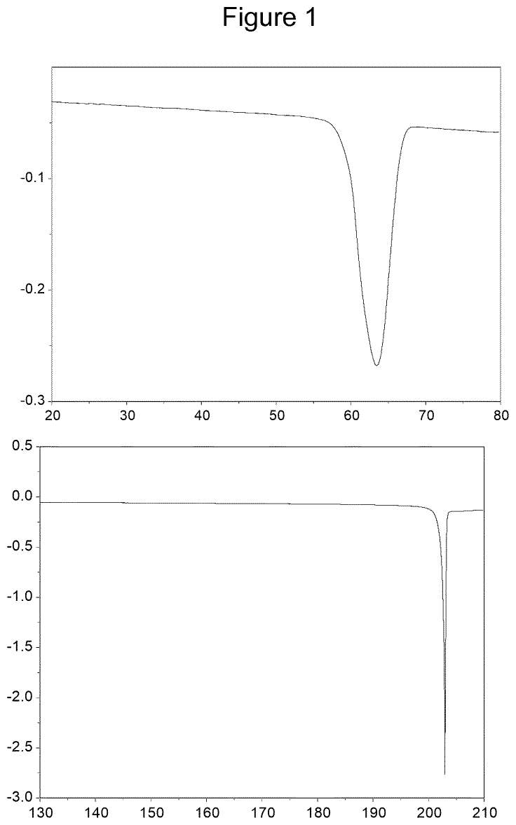

The crystalline base and the crystalline hydrogen fumarate salt of compound (Ia) have melting points around 60° C. and 203° C., respectively (see FIG. 1). Hence, according to the “Beaman's rule” the amorphous base and the amorphous hydrogen fumarate salt of compound (Ia) are exspected to have glass transition temperatures about −51° C. and 44° C., respectively (as shown in example 1). This indicates that these materials are only anticipated to remain substantially in the amorphous state at very low temperatures (50 K below Tg); i.e. below −6° C. or 267 K for the hydrogen fumarate and below −100° C. or 173 K for the base.

### Amorphous Compounds of Formula (I) and Amorphous Compounds of Formula (I) Salts

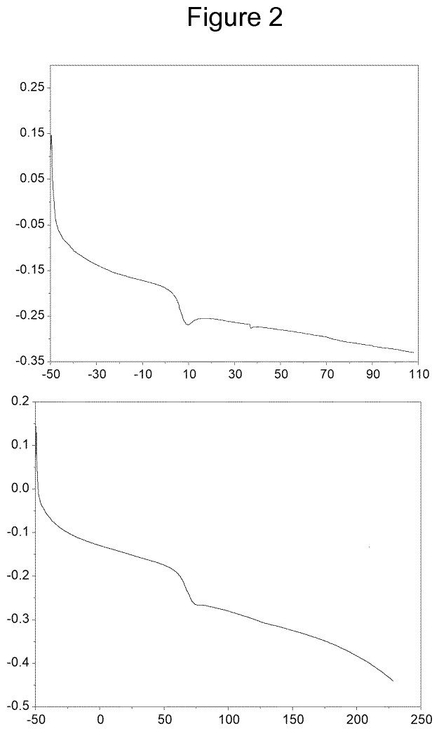

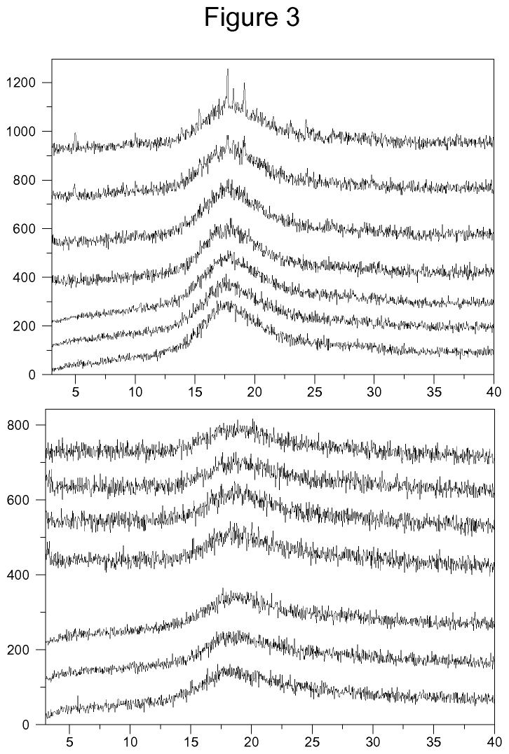

The inventors of the present invention have found that amorphous compounds of Formula (I), does not convert to any crystalline form upon storage at ambient conditions for up to 5 months. The measured glass transition temperatures of the amorphous base and the amorphous hydrogen fumarate salt of Compound (la) proved to be higher than calculated by the “Beaman's rule”. The actual Tg values were about 4° C. for the amorphous base and 66° C. for the amorphous hydrogen fumarate salt (see FIG. 2). But even if the actual glass transition temperatures are considered, it is still surprising that the amorphous compound (I) forms remains in the amorphous state at ambient conditions for several months (see FIG. 3).

In the present invention, compounds of Formula (I) is utilized as either crystalline free base or as a crystalline salt prior to being processed into the amorphous solid form, as described hereinbelow. Such salts may be prepared in a conventional manner known in the art e.g. by treating a solution or suspension of a free base compound (I) with a molar equivalent of a pharmaceutically acceptable acid. Representative examples of suitable organic and inorganic acids are given in the section defining the term “salts”.

### Methods for Obtaining the Amorphous Compounds of Formula (I) and Amorphous Compounds of Formula (I) Salts

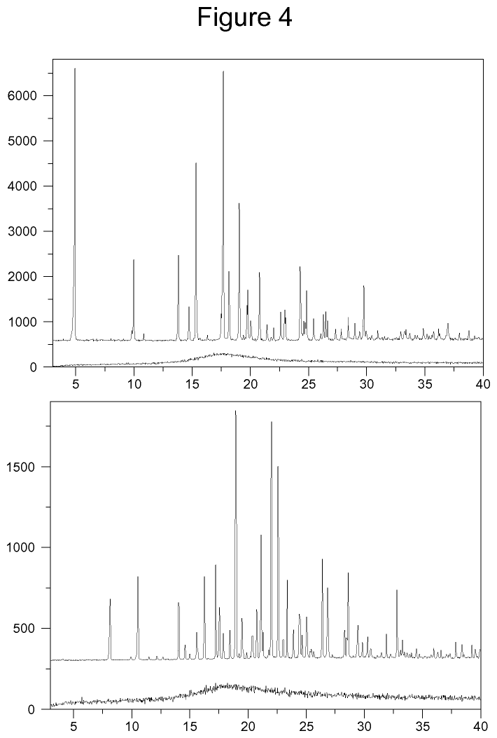

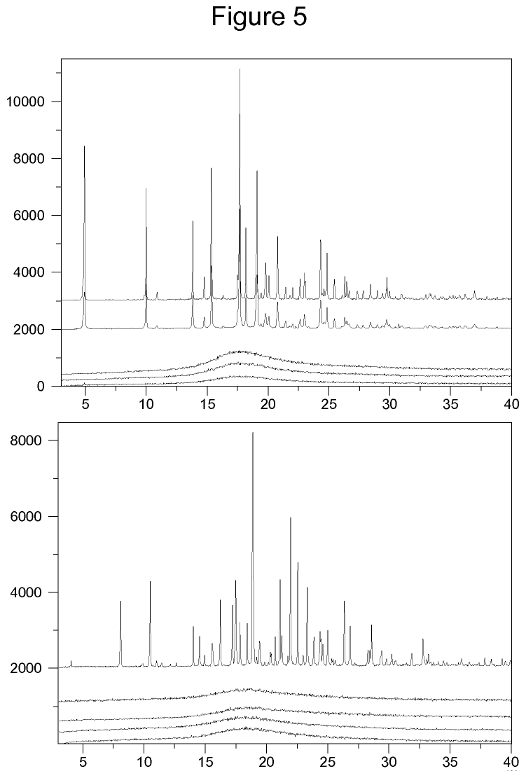

The unexpected stability of amorphous compounds of Formula (I) enabled the production of stable amorphous solids by conventional techniques based on melting of the pure crystals, e.g. melt quenching (FIGS. 4 and 5), which was not anticipated considering the low glass transition temperature.

The invention provides amorphous compounds of Formula (I) or amorphous compounds of Formula (I) salts obtainable by conventional techniques. These techniques include mechanical activation, solvent-based and melting-based methods (FIG. 5). The methods are further described in the experimental section; examples 5-8.

One illustrative process comprises applying mechanical stress to crystalline compound (I) and hereby induce defects in the crystal lattice, which eventually may manifest throughout the entire crystal with the subsequent loss of the long-range order, resulting in partial or full amorphization. The mechanical stress may be applied by any technique known in the art e.g. ball milling or cryo-milling. In FIG. 5 it is shown that only the hydrogen fumarate salt of compound (Ia) and not the base of compound (Ia) can successfully be turned into amorphous solid form upon ball milling. This finding is likely connected to the very low melting point of the crystalline base of compound (Ia), which hampers the mechanical activation process because the particles are not only mechanically activated during ball milling but also melted due to the heat generated by the process.

One additional illustrative process comprises (a) the preparation of a solution of compound (I) or a compound (I) salt in one or more solvents; and (b) removing the solvent(s) to obtain a substantially amorphous compound (I) or a substantially amorphous compound (I) salt. Removal of solvent(s) can be performed by any technique known in the art, e.g. by filmcasting, spray drying or freeze drying. This process optionally further comprises (c) a step of grinding the obtained substantially amorphous compound (I) or substantially amorphous compound (I) salt resulting from step (b) to form a finer grain powder more suitable for further pharmaceutical formulation processes. Non-limiting examples of suitable solvents for use in such processes are: Acetone, Water, Tert-butanol (TBA), Methanol (MeOH), Tetrahydrofuran (THF), Ethanol (EtOH), Trifluoroethanol (TFE), Acetonitrile (ACN), 2-Propanol, Dioxane, Ethylene diamine, Dimethylformamide (DMF), Glycerine, Propylene glycol, Dimethyl sulfoxide (DMSO), Ethylene glycol, Formamide, and Triethylene glycol etc. In the processes described above solvents can be used at different water:solvent or solvent:solvent ratios e.g. acetonitrile:water (50:50 v/v).

Another illustrative process comprises (a) a step of melting crystalline compound (I) or a crystalline compound (I) salt; and (b) a step of rapidly cooling the melt to form a substantially amorphous compound (I) or a substantially amorphous compound (I) salt. Melting step (a) can be performed by any technique known in the art, for example, by heating the compound in an oven to temperatures above its melting temperature. Cooling step (b) can be performed by any suitable method, for example by removing the heated material from the heat source and optionally further cooling the melt in a freezer or in liquid nitrogen. This process optionally further comprises (c) a step of grinding the obtained substantially amorphous compound (I) or substantially amorphous compound (I) salt resulting from step (b) to form a finer grain powder more suitable for further pharmaceutical formulation processes.

The optional grinding step (c) as mentioned hereinabove can be performed by any suitable method, for example by grinding in a mortar and pestle or by grinding in a mill.

Amorphous compounds of Formula (I) or amorphous compounds of Formula (I) salts of the present invention can be prepared by any suitable process, not limited to processes described herein.

### Crystallization Inhibition of Amorphous Compounds of Formula (I) and Amorphous Compounds of Formula (I) Salts

To further process and stabilize the amorphous form of compounds of Formula (I) and obtain a composition suitable for pharmaceutical formulation, the amorphous solid can be stabilized by one or more crystallization inhibitors. Known techniques in the art, e.g. melt based, mechanical activation or solvent based methods, may be used for obtaining solid dispersions, co-amorphous compositions, amorphous complexes and adsorption compositions comprising the compound of Formula (I) and such crystallization inhibitors. The exact mechanism of which the stabilization of the amorphous form is obtained is not known in details, but it is believed to be linked to the spatial separation of the drug molecules as well as the possibility to establish hydrogen bonds between the drug and the crystallization inhibitors.

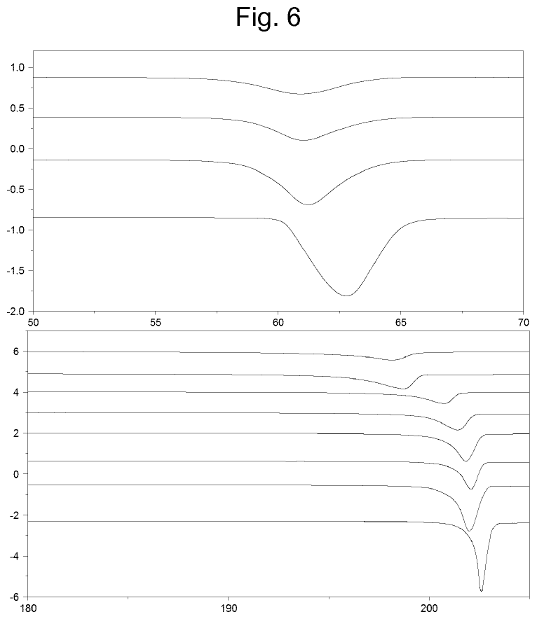

In the present invention, solid dispersions contain a compound of Formula (I) and one or more polymers and/or co-polymers. Examples of such solid dispersions suitable for further pharmaceutical formulations are presented in FIG. 6, table 1 and in example 9. Non-limiting examples of polymers and co-polymers that may be suitable as crystallization inhibitors are: Dimethylaminoethyl methacrylate, butyl methacrylate, and methyl methacrylate in a ratio of about 2:1:1 (e.g. Eudragit® EPO), VP/VA copolymer (60:40 linear random copolymer of N-vinyl-2-pyrrolidone and vinyl acetate, e.g. Plasdone™ S-630), PVP (Polyvinylpyrrolidone; e.g. Plasdone™ K12 and Plasdone™ K25), HPMC (hydroxypropyl methylcellulose, e.g. Pharmacoat® 603), HPMCAS (hypromellose-Acetate-Succinate, e.g. AQOAT AS MF), polyvinyl caprolactam polyvinyl acetate-polyethylene glycol graft copolymer (e.g. Soluplus®), anionic methacrylate copolymer with an average molecular weight of 280.000 Da (e.g. Eudragit® FS 100), ammonio methacrylate copolymer, polyvinyl acetate phthalate (e.g. Sureteric®), methyl cellulose, polyvinylpolypyrrolidone, poly(oxyethylene), Poly(ethyl acrylate-co-methyl methacrylate-co-trimethylammonioethyl methacrylate chloride), hydroxypropyl methyl cellulose phthalate, Poly(acrylic acid), Poly(ethylene glycol)-block-poly(propylene glycol)-block-poly(ethylene glycol), acetylated chitin, Poly(D-glucosamine), Macrogol-poly(vinyl alcohol) graft-copolymer, Polyvinyl alcohol-polyethylene glycol graft-copolymer.

The invention further relates to, co-amorphous compositions comprising amorphous low molecular weight molecules, e.g. amino acids and compounds of Formula (I), wherein the low molecular weight molecule is suitable to stabilize the amorphous state of the compound of Formula (I). Non-limiting examples of such molecules are: L-tryptophan, L-phenylalanine, L-methionine, L-valine, L-lysine, L-leucine.

Amorphous complexes in the present invention refers to complexes containing compounds of Formula (I) and cyclodextrins suitable to stabilize the amorphous state of the compound of Formula (I). Non-limiting examples of cyclodextrins are: Polyanionic β-cyclodextrin (e.g. Captisol®), Hydroxyl propyl-β-cyclodextrin (e.g. Kleptose® HPB), γ-cyclodextrin.

In the context of the present invention, adsorption compositions contain compounds of Formula (I) loaded into mesoporous silica suitable to stabilize the amorphous state of the compound of Formula (I). Examples of such solid dispersions suitable for further pharmaceutical formulations are presented in table 2 and in example 10. Non-limiting examples of mesoporous silicas are: Silica anhydrous (e.g. Aerosil® 300 and Aeroperl® 300), aluminium magnesium silicate (e.g. Neusilin® UFL2 and Neusilin® US2), SiO₂ (e.g. Parteck® SLC 500, Syloid® 72 FP, Syloid® 244 FP and Syloid® XDP 3050). These silicas have the following properties: Particle size: about 4-90 μm, Pore volume: about 0.56-1.70 cm³/g, Pore diameter: about 6-35 nm, and surface area: about 235-404 m²/g.

### Pharmaceutical Compositions Comprising Amorphous Compounds of Formula (I) or Amorphous Compounds of Formula (I) Salts

The present invention relates to compositions, and in particular to pharmaceutical compositions, comprising amorphous compound (I) or amorphous compound (I) salts and one or more pharmaceutically acceptable excipients. In a preferred aspect of the invention the pharmaceutical compositions further comprise one or more crystallization inhibitors.

The present invention also provides a process for making pharmaceutical compositions comprising amorphous compounds of Formula (I) or amorphous compounds of Formula (I) salts. The pharmaceutical compositions according to the invention may be obtained by mixing amorphous compounds of Formula (I) or amorphous compounds of Formula (I) salts with pharmaceutically acceptable excipients in accordance with conventional techniques such as those disclosed in Remington, The Science and Practice of Pharmacy, 22th edition (2012), Edited by Allen, Loyd V., Jr. Such excipients could be added to a pharmaceutical composition to improve its processing or storage properties. The excipient could also serve to facilitate formation of a dose unit of the composition into a pharmaceutically acceptable dosing unit, such as a capsule or tablet suitable for oral administration. Excipients employed in pharmaceutical compositions of the invention can be solid or liquid or a combination of both solid and liquid excipients.

An illustrative process comprises (a) a step of blending an amorphous compound of Formula (I), an amorphous compound of Formula (I) salt, or a composition comprising an amorphous compound of Formula (I) of the invention with one or more excipients to form a pharmaceutical composition, and (b) a step of tableting or encapsulating the blend or mix to form tablets or capsules, respectively. In a preferred embodiment of the invention, the pharmaceutical compositions for oral administration include solid oral dosage forms such as tablets, capsules, powders and granules.

Examples of excipients suitable for solid oral formulation include, but are not limited to, micro-crystalline cellulose, corn starch, lactose, mannitol, povidone, croscarmellose sodium, sucrose, cyclodextrin, talcum, gelatin, pectin, magnesium stearate, stearic acid and lower alkyl ethers of cellulose. Similarly, the solid formulation may include excipients for delayed or ex-tended release formulations known in the art, such as glyceryl monostearate or hypromellose. The amount of solid excipient will vary widely but will typically range from about 25 mg to about 1 g per dosage unit.

Further excipients may be used in solid oral formulations, such as colorings, flavorings and preservatives etc.

Other types of pharmaceutical compositions include suppositories, inhalants, creams, gels, dermal patches, implants and formulations for buccal or sublingual administration. It is requisite that the excipients used for any pharmaceutical formulation comply with the intended route of administration and are compatible with the active ingredients.

Pharmaceutical compositions of the invention contain a desired amount of Compound (I) per dose unit and, if intended for oral administration, can be in the form, e.g. of a tablet, a caplet, a pill, a hard or soft capsule, a lozenge, a cachet, a dispensable powder, granules or any other form adapted for such administration. Oral dosage forms that are discrete dose units each containing a predetermined amount of the drug, such as tablets or capsules are preferred.

The dosage forms of the invention can be prepared by any suitable process known in the art and are not limited to processes described herein.

### Use of Amorphous Compounds of Formula (I) and Amorphous Compounds of Formula (I) Salts

The present invention relates to stable novel solid forms of compound (I) for use in the treatment of central nervous system (CNS) disorders, e.g. psychiatric disorders such as schizophrenia, including Treatment Resistant Schizophrenia (TRS).

Amorphous compounds of Formula (I), or amorphous compounds of Formula (I) salts, or pharmaceutical composition as described hereinabove can be administered by any suitable route of administration. preferably, such routes comprise the oral, rectal, nasal, buccal, sublingual, transdermal and parenteral (e.g. subcutaneous, intramuscular, and intravenous) route; the oral route being the most preferred. It is appreciated that the route will depend on the general condition and age of the subject to be treated, as well as the nature of the condition to be treated and the properties of the active ingredient.

The present invention also relates to the medical use of amorphous compounds of Formula (I), such as for the treatment of a disease in the central nervous system, including psychosis, in particular schizophrenia, including treatment resistant schizophrenia, or other diseases involving psychotic symptoms, such as, e.g., Schizophreniform Disorder, Schizoaffective Disorder, Delusional Disorder, Brief Psychotic Disorder, Shared Psychotic Disorder as well other psychotic disorders or diseases that present with psychotic symptoms, e.g. bipolar disorder, such as mania in bipolar disorder.

Amorphous compounds and/or compositions of the invention can further be used in treatment of disorders such as those described in, for example, U.S. Pat. Nos. 5,807,855; 7,648,991; 7,767,683; 7,772,240; 8,076,342; U.S. Patent Publication Nos. 2008/0269248; 2010/0069676; 2011/0178094; 2011/0207744; WO 2005/016900; EP 0 638 073; and J. Med. Chem. 1995, 38, 4380-4392; each herein incorporated by reference in its entirety.

Amorphous compounds of Formula (I) or amorphous compounds of Formula (I) salts, or pharmaceutical composition of the present invention as mentioned hereinabove, may be useful for treatment of CNS disorders, specifically psychiatric disorders, more specifically psychotic disorders such as schizophrenia, including Treatment Resistant Schizophrenia (TRS); optionally the amorphous compound (I) can be administered in combination with a neuroleptic agent selected from sertindole, olanzapine, risperidone, quetiapine, aripiprazole, haloperidol, clozapine, ziprasidone and osanetant.

The invention also relates to the medical use of amorphous compounds of the present invention as combination therapy in conjunction with other therapeutic agents such as those described in, for example, U.S. Pat. Nos. 5,807,855; 7,648,991; 7,767,683; 7,772,240; 8,076,342; U.S. Patent Publication Nos. 2008/0269248; 2010/0069676; 2011/0178094; 2011/0207744; WO 2005/016900; EP 0 638 073; and J. Med. Chem. 1995, 38, 4380-4392; each herein incorporated by reference in its entirety.

### Doses

The amorphous compound of the present invention can be administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of an amorphous compound of the present invention, such as 1-500 mg/day, such as 1-100 mg/day, 1-50 mg/day, 1-20 mg/day or 10-20 mg/day. Conveniently, the amorphous compounds of the invention are administered in a unit dosage form, as described hereinabove, containing said compounds in an amount of about 0.1 to 500 mg, such as 10 mg, 20 mg, 50 mg 100 mg, 150 mg, 200 mg or 250 mg.

### Embodiments

The following embodiments describes the invention in further detail. The embodiments are numbered consecutively, starting from number 1.
1. A composition comprising amorphous compounds of Formula (I) or amorphous compounds of Formula (I) salts

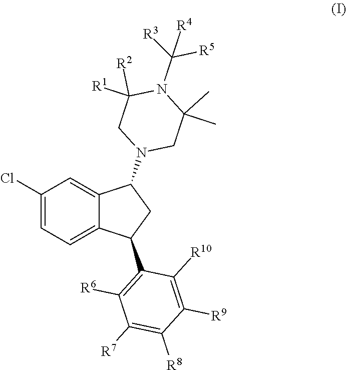

wherein, R1-R10 are independently selected from hydrogen or deuterium.
2. A composition according to embodiment 1, wherein at least one of R1-R10 is deuterium.
3. A composition according to embodiment 2, wherein R6-R10 are each deuterium.
4. A composition according to embodiment 3, wherein R3-R5 are each hydrogen.
5. A composition according to embodiment 3, wherein said amorphous compound or said amorphous compound salt is of Formula (Ia):

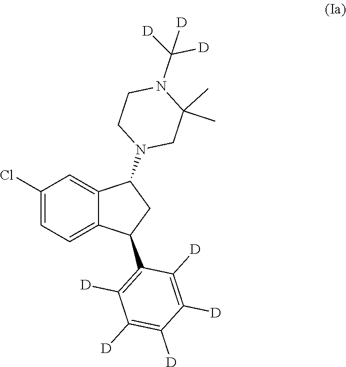 [SMILES: [2H]c1c([2H])c([2H])c([C@@H]2C[C@@H](N3CCN(C([2H])([2H])[2H])C(C)(C)C3)c3cc(Cl)ccc32)c([2H])c1[2H]]

6. A composition according to embodiment 5, wherein the amorphous compound of Formula (Ia) is the amorphous base.
7. A composition according to embodiment 5, wherein the amorphous compound of Formula (Ia) salt is the amorphous compound of Formula (Ia) hydrogen fumarate.
8. A composition of any of the preceding embodiments, wherein the amorphous compound remains substantially amorphous under ambient conditions for a period of 5 months.
9. A composition of any of the preceding embodiments, wherein the amorphous compound has a calculated glass transition temperature of 50 degrees Celsius or below, such as between −60 and 50 degrees Celsius, or between 0 and 50 degrees Celsius, or between 10 and 50 degrees Celsius, or between 20 and 50 degrees Celsius and remains substantially amorphous under ambient conditions for a period of 5 months.
10. A process for obtaining amorphous compounds of Formula (I) or amorphous compounds of Formula (I) salts comprising:
   a. preparing a solution of compounds of Formula (I) or a compounds of Formula (I) salt in one or more solvents; and
   b. obtaining amorphous compounds of Formula (I) or the amorphous compounds of Formula (I) salt by removing the solvent(s).
11. A process according to embodiment 9, wherein the solvent(s) are removed by spray-drying.
12. A process according to embodiment 9, wherein the solvent(s) are removed by freeze-drying.
13. A process according to embodiment 9, wherein the solvent(s) are removed by filmcasting.
14. A process according to any of embodiments 10-13, wherein the solvent(s) are selected from the group consisting of Acetone, Water, Tert-butanol (TBA), Methanol (MeOH), Tetrahydrofuran (THF), Ethanol (EtOH), Trifluoroethanol (TFE), Acetonitrile (ACN), 2-Propanol, Dioxane, Ethylene diamine, Dimethylformamide (DMF), Glycerine, Propylene glycol, Dimethyl sulfoxide (DMSO), Ethylene glycol, Formamide, and Triethylene glycol.
15. A process according to embodiment 13, wherein the solvent(s) are selected from the group consisting of Water, Tert-butanol (TBA), and Acetonitrile (ACN).
16. A process for obtaining amorphous compounds of Formula (I) or amorphous compounds of Formula (I) salts comprising:
   a. melting a crystalline compound of Formula (I) or a crystalline compound of Formula (I) salt;
b. obtaining an amorphous compound of Formula (I) or an amorphous compound of Formula (I) salt by rapidly cooling the melt.
17. A process according to embodiment 16, wherein the process is melt-quenching.
18. A process for obtaining amorphous compounds of Formula (I) or amorphous compounds of Formula (I) salts comprising:
   a. Application of mechanical stress to crystalline compound of Formula (I) or a crystalline compound of Formula (I) salt, to obtain an amorphous compound of Formula (I) or an amorphous compound of Formula (I) salt.
19. A process according to embodiment 18, wherein the mechanical stress is applied by ball-milling, and wherein the compound of Formula (I) is the hydrogen fumarate.
20. A process according to any of embodiment 10-19, wherein at least one of R1-R10 in the compound of Formula (I) is deuterium.
21. A process according to embodiment 20, wherein R6-R10 in the compound of Formula (I) are each deuterium.
22. A process according to embodiment 21, wherein R3-R5 in the compound of Formula (I) are each hydrogen.
23. A process according to embodiment 21, wherein said amorphous compound or said amorphous compound salt is of Formula (Ia).
24. A process according to any of embodiments 10-18, wherein the compound of Formula (Ia) is the base.
25. A process according to embodiment 23, wherein the compound of Formula (Ia) salt is the compound of Formula (Ia) hydrogen fumarate.
26. A composition comprising:
   a. an amorphous compound of Formula (I) or an amorphous compound of Formula (I) salts; and
   b. at least one crystallization inhibitor in an amount effective to reduce, delay or eliminate the formation of crystalline particles in the amorphous solid.
27. The composition of embodiment 26 wherein the crystallization inhibitor is a polymer or a co-polymer.
28. The composition of embodiment 27 wherein the polymer or copolymer is selected from the group consisting of Hypromellose Acetate Succinate (e.g. AQOAT AS MF), hydroxypropyl methylcellulose (e.g. Pharmacoat® 603), anionic methacrylate copolymer with an average molecular weight of 280.000 Da (e.g. Eudragit® FS100), polyvinyl caprolactam polyvinyl acetate-polyethylene glycol graft copolymer (e.g. Soluplus®), VP/VA; 60:40 linear random copolymer of N-vinyl-2-pyrrolidone and vinyl acetate (e.g. Plasdone S-630), Dimethylaminoethyl methacrylate, butyl methacrylate, and methyl methacrylate in a ratio of about 2:1:1 (e.g. Eudragit® EPO) and polyvinylpyrrolidone (e.g. Plasdone™ K12).
29. The composition of embodiment 28 wherein the copolymer or copolymer is selected from the group comprising polyvinyl caprolactam polyvinyl acetate-polyethylene glycol graft copolymer (e.g. Soluplus®), Dimethylaminoethyl methacrylate, butyl methacrylate, and methyl methacrylate in a ratio of about 2:1:1 (e.g. Eudragit® EPO) and polyvinylpyrrolidone (e.g. Plasdone™ K12).
30. The composition of embodiment 26 wherein the crystallization inhibitor is a cyclodextrin.
31. The composition of embodiment 30 wherein the cyclodextrin is selected from the group comprising Polyanionic β-cyclodextrin (e.g. Captisol®), Hydroxyl propyl-β-cyclodextrin (e.g. Kleptose® HPB) and γ-cyclodextrin.
32. The composition of embodiment 26 wherein the crystallization inhibitor is an amino acid.
33. The composition of embodiment 32 wherein the amino acid is selected from the group comprising L-tryptophan, L-phenylalanine, L-methionine, L-valine, L-lysine, L-leucine.
34. The composition of embodiment 26 wherein the crystallization inhibitor is mesoporous silica.
35. The composition of embodiment 34, wherein the mesoporous silica has a particle size of about 4-90 μm, a pore volume of about 0.56-1.70 cm³/g, a pore diameter of about 6-35 nm, and a surface area of about 235-404 m²/g.
36. The composition of embodiment 35 wherein the mesoporous silica is selected from the group comprising Silica anhydrous (e.g. Aerosil® 300 and Aeroperl® 300), aluminum magnesium silicate (e.g. Neusilin® UFL2 and Neusilin® US2), SiO₂ (e.g. Parteck® SLC 500, Syloid® 72 FP, Syloid® 244 FP and Syloid® XDP 3050).
37. A composition according to any of embodiment 26-36, wherein at least one of R1-R10 in the compound of Formula (I) is deuterium.
38. A composition according to embodiment 37, wherein R6-R10 in the compound of Formula (I) are each deuterium.
39. A composition according to embodiment 38, wherein R3-R5 in the compound of Formula (I) are each hydrogen.
40. A composition according to embodiment 38, wherein said amorphous compound or said amorphous compound salt is of Formula (Ia).
41. A composition according to embodiment 40, wherein the compound of Formula (Ia) is the base.
42. A composition according to embodiment 40, wherein the compound of Formula (Ia) salt is the compound of Formula (Ia) hydrogen fumarate.
43. A process for preparing a pharmaceutical composition, the process comprising:
   a. blending an amorphous compound of Formula (I) or an amorphous compound of Formula (I) salt; a
   b. crystallization inhibitor, and optionally
   c. one or more excipients
44. The process of embodiment 43 further comprising a grinding step of the obtained composition to obtain a solid more suitable for pharmaceutical formulation
45. A process according to embodiment 43-44 wherein the process further comprises tableting or encapsulating the composition to form tablets or capsules, respectively.
46. A process according to any of embodiment 43-45, wherein at least one of R1-R10 in the compound of Formula (I) is deuterium.
47. A process according to embodiment 46, wherein R6-R10 in the compound of Formula (I) are each deuterium.
48. A process according to embodiment 47, wherein R3-R5 in the compound of Formula (I) are each hydrogen.
49. A process according to embodiment 47, wherein said amorphous compound or said amorphous compound salt is of Formula (Ia).
50. A process according to embodiment 49, wherein the compound of Formula (Ia) is the base.
51. A process according to embodiment 49, wherein the compound of Formula (Ia) salt is the compound of Formula (Ia) hydrogen fumarate.
52. An amorphous compound of Formula (I) or a pharmaceutically acceptable salt thereof for use as a medicament.
53. An amorphous compound according to embodiment 52, wherein at least one of R1-R10 in the compound of Formula (I) is deuterium.
54. An amorphous compound according to embodiment 53, wherein R6-R10 in the compound of Formula (I) are each deuterium.
55. An amorphous compound according to embodiment 54, wherein R3-R5 in the compound of Formula (I) are each hydrogen.
56. An amorphous compound according to embodiment 54, wherein said amorphous compound or said amorphous compound salt is of Formula (Ia).
57. An amorphous compound according to embodiment 56, wherein the compound of Formula (Ia) is the base.
58. An amorphous compound according to embodiment 56, wherein the compound of Formula (Ia) salt is the compound of Formula (Ia) hydrogen fumarate.
59. The composition of any of embodiments 1-9 or 26-42, wherein the composition is a pharmaceutical composition.
60. Use of an amorphous compound of Formula (I) or an amorphous compound of Formula (I) salt or any of the compositions of embodiment 1-9 or 26-42 or 59 in the manufacture of a medicament, the medicament can for example be used in the treatment of psychosis, other diseases involving psychotic symptoms, psychotic disorders or diseases that present with psychotic symptoms.
61. Use of an amorphous compound of Formula (I) or an amorphous compound of Formula (I) salt or any of the compositions of embodiment 1-9 or 26-42 or 59 in the manufacture of a medicament according to embodiment 60, wherein the psychosis or disease involving psychotic symptoms is schizophrenia, including treatment resistant schizophrenia, schizophreniform disorder, schizoaffective disorder, delusional disorder, brief psychotic disorder, shared psychotic disorder, bipolar disorder or mania in bipolar disorder.
62. Use of an amorphous compound of Formula (I) or an amorphous compound of Formula (I) salt or any of the compositions of embodiment 1-9 or 26-42 or 59 in the manufacture of a medicament according to embodiment 61, wherein the disease involving psychotic symptoms is schizophrenia, including treatment resistant schizophrenia.
63. Use of an amorphous compound of Formula (I) or an amorphous compound of Formula (I) salt or any of the compositions of embodiment 1-9 or 26-42 or 59 in the manufacture of a medicament according to embodiment 62, wherein the disease involving psychotic symptoms is treatment resistant schizophrenia.
64. Use according to embodiment 60-63 wherein the amorphous compound of Formula (I) or the amorphous compound of Formula (I) salt or any of the compositions of embodiment 1-9 or 26-42 or 59 is administered in combination with one or more further compounds selected from the group consisting of sertindole, olanzapine, risperidone, quetiapine, aripiprazole, haloperidol, clozapine, ziprasidone and osanetant.
65. An amorphous compound of Formula (I) or an amorphous compound of Formula (I) salt or any of the compositions of embodiment 1-9 or 26-42 or 59 for use in the treatment of psychosis, other diseases involving psychotic symptoms, psychotic disorders or diseases that present with psychotic symptoms.
66. An amorphous compound of Formula (I) or an amorphous compound of Formula (I) salt or any of the compositions of embodiment 1-9 or 26-42 or 59 for use according to embodiment 65, wherein the psychosis or disease involving psychotic symptoms is schizophrenia, including treatment resistant schizophrenia, schizophreniform disorder, schizoaffective disorder, delusional disorder, brief psychotic disorder, shared psychotic disorder, bipolar disorder or mania in bipolar disorder.
67. An amorphous compound of Formula (I) or an amorphous compound of Formula (I) salt or any of the compositions of embodiment 1-9 or 26-42 or 59 for use according to embodiment 66, wherein the disease involving psychotic symptoms is schizophrenia, including treatment resistant schizophrenia.
68. An amorphous compound of Formula (I) or an amorphous compound of Formula (I) salt or any of the compositions of embodiment 1-9 or 26-42 or 59 for use according to embodiment 67, wherein the disease involving psychotic symptoms is treatment resistant schizophrenia.
69. An amorphous compound of Formula (I) or an amorphous compound of Formula (I) salt or any of the compositions of embodiment 1-9 or 26-42 or 59 for use according to embodiment 65, wherein said compound or composition is administered in combination with one or more further compounds selected from the group consisting of sertindole, olanzapine, risperidone, quetiapine, aripiprazole, haloperidol, clozapine, ziprasidone and osanetant.
70. A method for the treatment of psychosis, other diseases involving psychotic symptoms, psychotic disorders or diseases that present with psychotic symptoms comprising the administration of a therapeutically effective amount of an amorphous compound of Formula (I) or an amorphous compound of Formula (I) salt or any of the compositions of embodiment 1-9 or 26-42 or 59 to a patient in need thereof.
71. The method described in embodiment 70, wherein the psychosis or disease involving psychotic symptoms is schizophrenia, including treatment resistant schizophrenia, schizophreniform disorder, schizoaffective disorder, delusional disorder, brief psychotic disorder, shared psychotic disorder, bipolar disorder or mania in bipolar disorder.
72. The method described in embodiment 71, wherein the disease involving psychotic symptoms is schizophrenia, including treatment resistant schizophrenia.
73. The method described in embodiment 72, wherein the disease involving psychotic symptoms is treatment resistant schizophrenia.

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXPERIMENTAL SECTION

The examples provided below serve to facilitate a more complete understanding of the invention. The following examples illustrate the exemplary modes of making and practicing the invention.

However, the scope of the invention is not limited to specific embodiments disclosed in these Examples, which are for purposes of illustration only, since alternative methods can be utilized to obtain similar results.

### Characterization of Amorphous Compounds of Formula (I) and Amorphous Compounds of Formula (I) Salts

### Example 1—Determining Glass Transition Temperatures

As stated in the description of the present application, it is generally recognized that the glass transition temperature can be estimated as ⅔ of the melting temperature of the crystalline material (measured in Kelvin). From the DSC thermograms in FIG. 1 (left), it is seen that the crystalline base of compound (Ia) has an endothermic peak around 60° C. corresponding to the melting of the crystals. Based on the melting temperature of the crystalline base of compound (la), the calculated glass transition temperature of the amorphous form of this compound is thus ⅔*(60° C.+273)=222 K or −51° C. However, when the actual Tg of the amorphous base of compound (Ia) was determined, it was seen that the corresponding thermogram showed a change in heat capacity around 5° C. corresponding to a glass transition (FIG. 2—left). This means that the actual measured Tg of this amorphous form is around 5° C. and not −51° C. as calculated using the Beaman Rule.

Similar experiments were performed with the hydrogen fumarate of compound (Ia). Here the calculated Tg was based on the observed endothermic peak around 203° C. corresponding to the melting of the crystal (FIG. 1—right). Consequently, the calculated Tg is ⅔ *(203° C.+273)=317 K or 44° C. The thermogram of the amorphous form is shown in FIG. 2 (right) and depicts a change in heat capacity around 66° C. corresponding to a glass transition. This means that the actual measured Tg of the amorphous hydrogen fumarate of compound (Ia) is around 66° C. and not 44° C. as calculated.

As seen in FIG. 2, none of the tested amorphous materials recrystallizes upon heating, but remains amorphous. This indicates that the amorphous base as well as the fumarate salt do not recrystallize easily above the glass transitions, which is indicative of very stable amorphous forms.

### Example 2—X-Ray Powder Diffraction (XRPD)

X-Ray powder diffractograms were measured on a PANalytical X′Pert PRO X-Ray Diffractometer using CuK_{α1} radiation (λ=1.5406 Å). The samples were measured in reflection mode in the 2θ-range 3-40° using an X'celerator detector. (See FIGS. 3, 4 and 5).

FIG. 4 displays the XRPD diffractograms belonging to the base and the hydrogen fumarate salt of compound (Ia) in crystalline and amorphous form. For this experiment the amorphous forms were prepared by melt-quenching and this technique is described in detail in example 5. The diffractogram of the base of compound (Ia) in crystalline form shows sharp and well-defined Bragg peaks at around 4.9, 10.0, 15.4, 17.7 and 19.1° 20, characteristic of the crystalline α-form. In contrast the diffraction pattern recorded after melt-quenching shows a diffuse halo with no Bragg peaks, characteristic of an amorphous material (FIG. 4—left).

The diffractogram of the hydrogen fumarate of compound (Ia) in crystalline form shows sharp and well-defined Bragg peaks at around 8.1, 10.5, 18.9 and 22.0° 20, characteristic of the crystalline α-form, whereas the diffraction pattern recorded after melt-quenching shows a diffuse halo with no Bragg peaks, characteristic of an amorphous material (FIG. 4—right).

### Example 3—Differential Scanning Calorimetry (DSC)

Different scanning programs are used depending on the purpose of the analysis as given in example 9. In all DSC-experiments, 2-5 mg sample is packed into Tzero aluminum hermetic pans with a perforated lid, and the measurement is performed under 50 mL/min dry nitrogen gas purge.

Melting point and glass transition determined on the base of compound (Ia): Sample of 2-3 mg was packed into Tzero aluminum hermetic pans with a perforated lid and heated 1° C./min to 80° C. (giving the melting point), kept isothermal for 2 min., equilibrated at −50° C., and heated 10° C./min to 110° C. (giving the glass transition of the formed amorphous solid).

Melting point and glass transition of the hydrogen fumarate of compound (Ia): Sample heated 1° C./min to 210° C. (giving the melting point), kept isothermal for 2 min., equilibrated at −50° C., and heated 10° C./min to 230° C. (giving the glass transition of the formed amorphous solid).

### Example 4—Physical Stability

The amorphous base and the fumarate salt of compound (Ia) were analyzed using XRPD to determine the physical stability of these solid forms. The presence of a substantially amorphous solid form was confirmed by a diffuse halo XRPD pattern similar to FIG. 3, which is characteristic for a solid amorphous form.

The short-term physical stability of the amorphous solids was analyzed immediately after preparation; the samples were analyzed by XRPD for 96 hours consecutively by alternating measurements every 2 hours. The long-term stability was then assessed by storing the amorphous solids under ambient conditions and analyzing them by XRPD after 1, 4 and 9 weeks as well as 5, 8 and 12 months. The characteristic XRPD patterns confirm substantially amorphous solids at all timepoints for the fumarate and weak indication of crystallization of the free base after 5 months are shown in FIG. 3.

### Processes for Obtaining Amorphous Compounds of Formula (I) and Amorphous Compounds of Formula (I) Salts

### Example 5—Melt-Quenching

Due to the low calculated Tg of the amorphous forms of Compound (I), it was not expected that it would be possible to produce an amorphous solid using conventional technique based on the melting point of the pure crystal; e.g. melt-quenching. However, despite the low calculated Tg it was possible to produce very stable amorphous forms by melt-quenching, which is seen in the XRPD diffractograms in FIGS. 3 and 4.

Amorphous base and amorphous hydrogen fumarate of Compound (la) were prepared from the corresponding crystalline forms; Approximately 5 mg crystalline solid was placed in the center of an XRPD plate or in a DSC pan and heated to approximately 5° C. above the melting temperature of the pure crystal (65° C. for the base of Compound (la) and 210° C. for the hydrogen fumarate) for 5 min in an electronic furnace. Subsequently, the melt was removed from the furnace. The resulting amorphous solids were characterized using XRPD and DSC as described above (FIGS. 1, 2, 3, 4 and 5).

### Example 6—Ball Milling

Approximately 500 mg crystalline base and crystalline hydrogen fumarate of compound (Ia) were weighed into two separate 25 mL stainless steel grinding jars each containing two 12 mm stainless steel ball bearings and milled at 30 Hz for a total of 15 min for the fumarate and 30 min for the base using a Mixer mill MM 400 from Retsch. The resulting solids were characterized using XRPD (FIG. 5).

The diffraction pattern recorded after ball milling of the crystalline hydrogen fumarate shows a diffuse halo with no Bragg peaks, characteristic of an amorphous material (FIG. 5—right). This specifies that it is possible to produce the hydrogen fumarate of compound (Ia) in amorphous form using mechanical activation preparation methods.

In contrast, the XRPD diffractogram for the base of compound (Ia) after ball milling reveals a Bragg peak pattern similar to that of the α-form of the solid form, which shows that the material is not fully amorphized during ball milling (FIG. 5—left). This specifies that ball milling is not the optimal method for preparing the amorphous form of the base of compound (Ia).

### Example 7—Spray Drying

Two separate solutions of 20 mg/ml crystalline base and crystalline hydrogen fumarate of compound (Ia) were prepared by dissolving 500 mg material in 25 ml methanol. The solutions were spray dried using an open-loop 4M8-TriX spray drier from ProCepT with air as drying gas. The process settings were as follows: 75° C. inlet air temperature, 0.50 m³/min inlet air volume, 50% pump speed and 20 L/min nozzle air. The resulting spray dried powder was then collected and analyzed using XRPD (FIG. 5).

### Example 8—Freeze Drying

Two separate 10 ml solutions of 50 mg/ml crystalline base and crystalline hydrogen fumarate of compound (Ia) were prepared using tert-butanol (TBA) and acetonitrile:water (50:50 v/v), respectively. TBA, acetonitrile and water are particularly suitable for freeze drying due to their high freezing temperature, low toxicity and high vapor pressure. However, due to the relatively high melting point of TBA around 26° C., heat is required to dissolve the solids in the solvent. Therefore, the solutions were prepared under stirring in a Variomag heated magnetic stirring block at 40° C. and 300 rpm. When a clear solution was formed, 200 μl was pipetted onto the center of a zero background (0-BG) XRPD silica plate (corresponding to 10 mg of compound (Ia)) and placed in a Styrofoam box containing dry ice until the sample had reached a temperature of approximately −80° C. The sample was then freeze dried in a DW 1,0—110 freeze dryer from Drawell Scientific by placing the frozen XRPD plate in the drying chamber and opening the valve to create a vacuum. When all solvent was sublimated and the pressure in the chamber had reached approximately 3·10⁻³ mbar, the freeze-drying process was terminated by slowly opening the valve and letting air enter the drying chamber and turning off the vacuum pump and a container was placed under the drain to collect the solvent. The resulting freeze dried powder was then collected and analyzed using XRPD (FIG. 5).

The diffraction patterns recorded after spray drying and freeze drying shows diffuse halos with no Bragg peaks, characteristic of an amorphous material. This specifies that it is possible to produce compounds of formula (I) in amorphous form using solvent based preparation methods.

### Crystallization Inhibitors

### Example 9—Amorphous Solid Dispersions Comprising Polymers and Co-Polymers and Compounds of Formula (I)

The crystalline solid form of compound (Ia) and various polymer or co-polymer mixtures in the ratios 90:10% or 85:15% (w/w) were prepared. The melting point of the mixtures were analyzed by DSC thermograms. Such analysis will confirm the ability of polymers and co-polymers to stabilize the amorphous compounds of Formula (I). As shown in the thermograms in FIG. 6, all mixtures demonstrated some degree of melting point depression compared to the crystalline form of compound (Ia). The crystalline base of compound (Ia) has a melting peak with an onset around 60° C., characteristic of the crystalline α-form. After mixing with 10% (w/w) anionic methacrylate copolymer with an average molecular weight of 280.000 Da (Eudragit® FS100), polyvinyl caprolactam polyvinyl acetate-polyethylene glycol graft copolymer (Soluplus®) and Dimethylaminoethyl methacrylate, butyl methacrylate, and methyl methacrylate in a ratio of about 2:1:1 (Eudragit® EPO), the onset of melting of the solid was depressed to 58.9, 58.8 and 58.5° C., respectively (FIG. 6—left).

The thermogram for crystalline hydrogen fumarate of compound (Ia) shows a melting peak with an onset around 203° C., characteristic of the crystalline α-form. After mixing with 15% (w/w) Hypromellose Acetate Succinate (AQOAT AS MF), hydroxypropyl methylcellulose (Pharmacoat® 603), anionic methacrylate copolymer with an average molecular weight of 280.000 Da (Eudragit® FS100), polyvinyl caprolactam polyvinyl acetate-polyethylene glycol graft copolymer (Soluplus®), VP/VA; 60:40 linear random copolymer of N-vinyl-2-pyrrolidone and vinyl acetate (Plasdone™ S-630), Dimethylaminoethyl methacrylate, butyl methacrylate, and methyl methacrylate in a ratio of about 2:1:1 (Eudragit® EPO) and polyvinylpyrrolidone (Plasdone™ K12), the onset of melting of the drug was depressed to 201.4, 201.4, 201.0, 199.7, 198.6, 196.0 and 195.2° C., respectively (FIG. 6—right).

Consequently, polyvinyl caprolactam polyvinyl acetate-polyethylene glycol graft copolymer (Soluplus®) and Dimethylaminoethyl methacrylate, butyl methacrylate, and methyl methacrylate in a ratio of about 2:1:1 (Eudragit® EPO) are particularly well suited for stabilizing the amorphous base of compound (Ia). Similarly, Dimethylaminoethyl methacrylate, butyl methacrylate, and methyl methacrylate in a ratio of about 2:1:1 (Eudragit® EPO) and polyvinylpyrrolidone (Plasdone™ K12) are particularly well suited for stabilizing the amorphous hydrogen fumarate of compound (Ia), however, all exemplified polymers showed potential to function as crystallization inhibitors for amorphous compounds of Formula (I) or amorphous compounds of Formula (I) salts.

The loading capacity if compounds of Formula (I) in various polymers were also determined:

Crystalline base or hydrogen fumarate of compound (I) and the various polymers or co-polymers (polyvinylpyrrolidone (Plasdone™ K12 and Plasdone™ K25), VP/VA copolymer (60:40 linear random copolymer of N-vinyl-2-pyrrolidone and vinyl acetate; Plasdone'S-630) and Dimethylaminoethyl methacrylate, butyl methacrylate, and methyl methacrylate in a ratio of about 2:1:1 (Eudragit® EPO)) were mixed in the following ratios; 70, 75, 80, 85, 90 and 95% (w/w) crystalline solid form in polymer. The resulting mixtures were transferred into a mortar followed by a thorough grinding using a pistil.

The loading capacity evaluation of the polymers utilizes DSC thermograms. Again, the melting point depression is considered, because it is a direct measure of the drug-polymer solubility. From this analysis, it is possible to predict the drug-polymer solubility at room temperature through extrapolation of the melting point of different compositions at elevated temperatures using the Flory-Huggins model:

$$\frac{Δ {H}_{m}}{R} \cdot ( \frac{1}{{T}_{m}} - \frac{1}{T} ) = ln ( {v}_{drug} ) + ( 1 - \frac{1}{λ} ) \cdot ( 1 - {v}_{drug} ) + χ \cdot {( 1 - {v}_{drug} )}^{2} ,$$

where ΔHm and Tm are the enthalpy of fusion and melting temperature for the crystalline form of compound (I), respectively. R is the gas constant, λ is the molar volume ratio of the polymer to the drug, χ is the Flory-Huggins interaction parameter, T is the temperature at which the measurement is made and v_{drug} is the volume fraction of the crystalline form of compound (I) in the polymer. Consequently, by plotting the onset of melting (T) and volume fraction of the crystalline form in the mixture (v_{drug}), the average fitting parameter χ can be calculated and through extrapolation of the Flory-Huggins model to T=25° C., the drug-polymer solubility at room temperature can be predicted.

The following results were obtained regarding the loading capacity of compound (Ia) in various polymers:

TABLE 1

| | Mean Loading |
|---|---|
| Compound (Ia)-Polymer | Capacity |
| Base-Polyvinylpyrrolidone (Plasdone ™ K12) | 7.2% |
| Base-VP/VA copolymer (60:40 linear random | 10.2% |
| copolymer of N-vinyl-2-pyrrolidone and vinyl | |
| acetate; Plasdone ™ S-630) | |
| Base-Dimethylaminoethyl methacrylate, butyl | 10.3% |
| methacrylate, and methyl methacrylate in a | |
| ratio of about 2:1:1 (Eudragit ® EPO) | |
| Hydrogen fumarate-Polyvinylpyrrolidone | 0.9% |
| (Plasdone ™ K25) | |
| Hydrogen fumarate-VP/VA copolymer (60:40 | 1.0% |
| linear random copolymer of N-vinyl-2- | |
| pyrrolidone and vinyl acetate; Plasdone ™ S-630) | |
| Hydrogen fumarate-Dimethylaminoethyl | 2.0% |
| methacrylate, butyl methacrylate, and methyl | |
| methacrylate in a ratio of about 2:1:1 | |
| (Eudragit ® EPO) | |

### Example 10—Adsorption Compositions Comprising Mesoporous Silicas and Compounds of Formula (I)

The nano-pores in the mesoporous silica particles have a size-constraining effect on nucleation and crystal growth. Thus, loading the drug into these pores may prevent crystallization in amorphous systems. To achieve a thermodynamically stable system, the drug overload in the mixture should be avoided and therefore, determining the maximum loading capacity is essential. Various mesoporous silicas were used in the following experiments: Silica anhydrous (Aeroperl® 300) and SiO₂ (Parteck® SLC 500 and Syloid® 72 FP).

The compounds of Formula (Ia) were mixed with the mesoporous silicas in ratios of 80% compound (Ia) and 20% silica (w/w). The mixtures were transferred into a mortar followed by thorough grinding using a pistil. The heat capacity change (ΔCp) over the glass transition temperature (Tg) of the pure compound (Ia) solids and compound (I)-silica physical mixtures were determined after a heat-cool-heat procedure with sample powders of −5 mg packed into Tzero aluminum hermetic pans with a perforated lid and annealed at 110° C. for 2 min (for mixtures containing the base of compound (Ia)) or 210° C. (for mixtures containing the fumarate of compound (Ia)) and cooled to −50° C. After quenching, the samples were ramped at a rate of 20° C./min to 90° C. or 230° C. under 50 mL/min dry nitrogen gas purge.

The maximum loading capacity of compound (Ia) in mesoporous silica were determined by extrapolation the linear trend to zero ΔCp. This represents the point where the maximum loading capacity is reached. The loading capacity of compound (Ia) in the three silicas were as follows:

TABLE 2

| | Mean Loading |
|---|---|
| Compound (Ia)-Mesoporous Silica | Capacity |
| Base-Silica anhydrous (Aeroperl ® 300) | 23.0% |
| Base-SiO₂ (Parteck ® SLC 500) | 32.6% |
| Base-SiO₂ (Syloid ® 72 FP) | 15.2% |
| Hydrogen fumarate-Silica anhydrous (Aeroperl ® 300) | 24.3% |
| Hydrogen fumarate-SiO₂ (Parteck ® SLC 500) | 31.0% |
| Hydrogen fumarate-SiO₂ (Syloid ® 72 FP) | 25.1% |

1. A composition comprising amorphous compounds of Formula (I) or amorphous compounds of Formula (I) salts 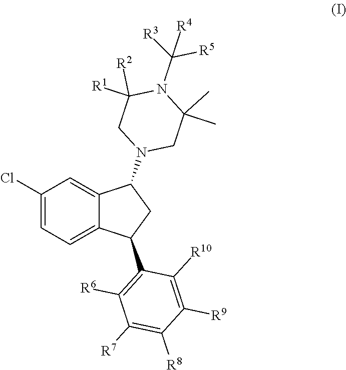
wherein, R1-R10 are independently selected from hydrogen or deuterium.

2. A composition according to claim 1, wherein at least one of R1-R10 is deuterium.

3. A composition according to claim 2, wherein R6-R10 are each deuterium.

4. A composition according to claim 3, wherein R3-R5 are each hydrogen.

5. A composition according to claim 3, wherein said amorphous compound or said amorphous compound salt is of Formula (Ia):

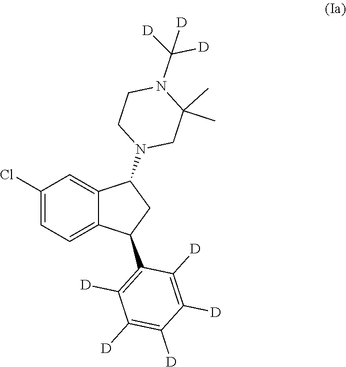 [SMILES: [2H]c1c([2H])c([2H])c([C@@H]2C[C@@H](N3CCN(C([2H])([2H])[2H])C(C)(C)C3)c3cc(Cl)ccc32)c([2H])c1[2H]]

6. A composition according to claim 5, wherein the amorphous compound of Formula (Ia) is the amorphous base.

7. A composition according to claim 5, wherein the amorphous compound of Formula (Ia) salt is the amorphous compound of Formula (Ia) hydrogen fumarate.

8. A process for obtaining amorphous compounds of Formula (I) or amorphous compounds of Formula (I) salts comprising:
a. preparing a solution of compounds of Formula (I) or a compounds of Formula (I) salt in one or more solvents; and
b. obtaining amorphous compounds of Formula (I) or the amorphous compounds of Formula (I) salt by removing the solvent(s).

9. A composition comprising:
a. an amorphous compound of Formula (I) or an amorphous compound of Formula (I) salts; and
b. at least one crystallization inhibitor in an amount effective to reduce, delay or eliminate the formation of crystalline particles in the amorphous solid.

10. The composition of claim 9, wherein the crystallization inhibitor is a polymer or a co-polymer.

11. The composition of claim 10, wherein the copolymer or copolymer is selected from the group comprising polyvinyl caprolactam polyvinyl acetate-polyethylene glycol graft copolymer (e.g. Soluplus®), Dimethylaminoethyl methacrylate, butyl methacrylate, and methyl methacrylate in a ratio of about 2:1:1 (e.g. Eudragit® EPO) and polyvinylpyrrolidone (e.g. Plasdone™ K12).

12. The composition of claim 9 wherein the crystallization inhibitor is mesoporous silica.

13. The composition of claim 12, wherein the mesoporous silica has a particle size of about 4-90 μm, a pore volume of about 0.56-1.70 cm³/g, a pore diameter of about 6-35 nm, and a surface area of about 235-404 m²/g.

14. (canceled)

15. A method for treating psychosis, other diseases involving psychotic symptoms, psychotic disorders or diseases that present with psychotic symptoms in a subject, comprising administering to the subject an amorphous compound of Formula (I) or an amorphous compound of Formula (I) salt: 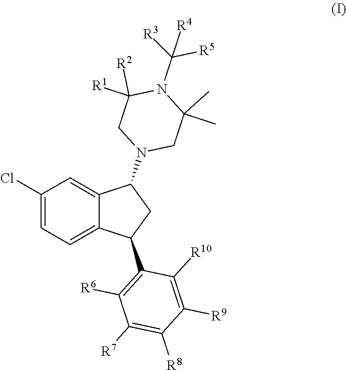
wherein, R1-R10 are independently selected from hydrogen or deuterium.

16. The method of claim 15, wherein the disease involving psychotic symptoms is schizophrenia, including treatment resistant schizophrenia.

17. A method for treating psychosis, other diseases involving psychotic symptoms, psychotic disorders or diseases that present with psychotic symptoms in a subject, comprising administering to the subject a composition of claim 1 to the subject.

18. A method for treating psychosis, other diseases involving psychotic symptoms, psychotic disorders or diseases that present with psychotic symptoms in a subject, comprising administering to the subject a composition of claim 9 to the subject.

19. A method for treating psychosis, other diseases involving psychotic symptoms, psychotic disorders or diseases that present with psychotic symptoms in a subject, comprising administering to the subject a composition of claim 5 to the subject.

20. A method for treating psychosis, other diseases involving psychotic symptoms, psychotic disorders or diseases that present with psychotic symptoms in a subject, comprising administering to the subject a composition of claim 7 to the subject.
